(19) United States
(12) Patent Application Publication
(10) Pub. No.: US 2017/0364657 A1
(43) Pub. Date: Dec. 21, 2017

(54) METHOD FOR EVALUATING A SCORE REPRESENTING THE HEALTH OF A PATIENT AND PRODUCTS IMPROVING THE SCORE

(71) Applicant: Michel GOLAY, Fourmex (CH)

(72) Inventor: Michel GOLAY, Fourmex (CH)

(21) Appl. No.: 15/541,490

(22) PCT Filed: Dec. 10, 2015
(86) PCT No.: PCT/IB2015/002318

§ 371 Date: Jul. 5, 2017

(30) Foreign Application Priority Data
Jan. 7, 2015 (CH) CH00014/15

(51) Int. Cl. G06F 19/00; A61K 36/899; A61K 36/81; A61K 36/752; A61K 36/73; A61K 36/23; A61K 36/20; A61K 36/185; A61K 31/732; A61K 31/19; A61K 36/906; A23L 33/105

(52) U.S. Cl. CPC G06F 19/3431; G06F 19/363; G06F 19/3475; A61K 36/906; A61K 36/899; A61K 36/81; A61K 36/752; A23L 33/105; A61K 36/23; A61K 36/20; A61K 36/185; A61K 31/732; A61K 31/19; A61K 36/73; A23V 2002/00

(57) ABSTRACT

A method for determining a numerical score representative of a patient's health, is characterized by the following steps. At an initial calibration step, a database is established from a series of indicators relating to the state of health of the patient, each indicator being assigned a numerical value, and afterwards a statistical analysis of this database is performed so as to establish for each of said indicators a score depending on a measured value of the indicators of the state of health with respect to reference values, and to establish at least four groups constituted by said indicators, each of said groups representing information corresponding respectively to oxidative stress, hereinafter Group 1; to functions of the digestive brain, hereinafter Group 2; to functions of the reptilian brain, hereinafter Group 3; and to physical abilities of the patient coupled to his information on his general state of health, hereinafter Group 4. Furthermore, a value is assigned to each group, then a health score S specific to the patient is calculated using the formula:

S=(Value Group 1+(2* Value Group 2)+(2* Value Group 3)+(3* Value Group 4))/(2* Number of groups)

An object of the present invention is a method for assessing a score representative of a patient's health in order to help him recover, optimize and maintain his health and ensure and enhance his well-being and longevity.

The immediate accessibility to the results of scientific research allows a phenomenal acceleration in the understanding of human physiology. Nevertheless, patients are still often treated according to erroneous or incomplete concepts. The present method bears only on recent and validated research in order to offer patients an optimal care.

Health is the fruit of the balance between two portions of the body, the digestive brain and the encephalic brain.

The digestive brain must ensure energy supplies, essential nutrients, vitamins and trace elements. It must also act as a protective barrier in the same way as the skin, the pulmonary mucosa and the blood—brain barrier, in order to prevent the inlet of undesired intruders. It permanently communicates with the brain via the enteric nervous system and by numerous hormones, lipoproteins, interleukins and other substances some of which are still unknown.

The digestive interface is to be considered in its entirety. Thousands of bacteria species which are contained in the digestive tract of an individual are a part thereof. They have been living in symbiosis with generations of individuals for thousands of years. Each individual feeds on the fruits of its metabolisms. They provide about 50% of the nutrients supplies essential to each individual. They also produce many lipoproteins, which are capable of sending information to the brain. They play a fundamental role in the immunity functions and especially in the production of inflammation, which may affect the entire body. Recent scientific evidence clearly demonstrates the links between intestinal flora and certain diseases, such as obesity or type-2 diabetes. Thus, it is fundamental to ensure the quality of the intestinal flora of an individual in order to preserve his health.

On the other hand, intestinal mucosa acts as a real barrier. It is constituted by juxtaposed cells which are held together by tight junctions. The integrity of these bonds is essential. The intestinal mucosa is the limit between the outside and the inside of the body. It is therefore not surprising that more than half the immunity defenses of an individual are located along his digestive tract. When a bacterium or a foreign protein penetrates through this protective layer, the immunitary system must take over for the individual to get rid of it. This may represent considerable energy expenses for the organism. This energy will no longer be available for other functions. Hence, it is crucial to ensure that the intestinal cells (enterocytes) receive an optimal nutrition and that nothing could attack the tight junctions.

The encephalic brain is the motor, regulator and coordinator of all essential functions. For this brain to operate well, it must on the one hand be protected by the digestive brain, and on the other hand it must be permanently activated. Physical activity, the movement through the stimulation of the neurons of the cerebellum, is the most important activation. The other visual, auditory, gustatory and tactile stimulations also play this role.

Throughout evolution, the brain has evolved. It is possible to distinguish different areas thereof depending on the stages of development. The primitive brain, called reptilian, is the most important area as regards health. It manages the essential functions and ensures the proper coordination of the different cycles setting the rhythm of an individual's days. The other areas have regulatory functions on the reptilian brain.

Hence, assessing the operation of all these areas is crucial if the individual wants to know the state of his health. This is done thanks to new discoveries made in the field of functional neurology. It is thus possible to stimulate or inhibit certain cerebral areas in order to modify their regulatory effects on the reptilian brain.

Moreover, the marker of cell suffering is well known. It consists of oxidative stress.

The present method aims to enable the determination of a score representative of the health of a patient so that both brains of an individual could operate in harmony and such that cells can be in an environment favorable to their development in particular by tackling the factors of oxidative stress.

In accordance with the invention, a method for determining a numerical score representative of the health of a patient, is characterized by the following steps. At an initial calibration step, a database is established from a series of indicators relating to the state of health of the patient, each indicator being assigned a numerical value. Afterwards, a statistical analysis of this database is performed so as to establish for each of said indicators a score depending on a measured value of the indicators of the state of health with respect to reference values, and to establish at least four groups constituted by said indicators, each of said groups representing information corresponding respectively to oxidative stress, hereinafter Group 1; to functions of the digestive brain, hereinafter Group 2; to functions of the reptilian brain, hereinafter Group 3; and to physical abilities of the patient coupled to his information on his general state of health, hereinafter Group 4. Furthermore, a value is assigned to each group, then a health score S specific to the patient is calculated using the formula:

S=Σ(Value Group 1+(2* Value Group 2)+(2* Value Group 3)+(3* Value Group 4))/(2* Number of groups)

In a preferred embodiment, the numerical score is determined on a scale of 0 to 100. The value of each group also varies from 0 to 100 whereas the value of each indicator varies from 0 to 10.

In a preferred embodiment, the value of Group 1 is determined using scores relating to a digestive analysis, an oxidized LDL (Low Density Lipoprotein) value, a HOMA (homoestasis model assessment) index, a Selenium value, a Ferritin value, and a Zinc value. Group 1 allows affecting a value to the oxidative stress. Oxidative stress is at the basis of numerous chronic diseases and is the result of very reactive molecules, linked to oxygen, that damage at every moment our most vital molecules and end by causing illness. The paradox is that these very reactive molecules, called free radicals, have important biological functions, by intervening in particular in cell signaling and are useful for the individual. When in excess, they may also attack all the constituents of a living organism and favor chronic diseases such as cataract, cancer, coronary artery diseases, diabetes, renal failure, Alzheimer, Parkinson . . . In theory, these free radicals are neutralized or taken up by protective mechanisms of the organism called antioxidants. Nowadays, antioxidants seem to be the key to longevity and allies for tackling modern diseases. They are protective elements which intervene by trapping free radicals. Oxidative stress may generate a physical or nervous fatigue, temporary or persistent, as well as eating disorders.

In one embodiment, the value of Group 2 is determined using scores relating to a digestive analysis, a CRP (C-reactive protein) value, a HOMA index and a value of the Omega 6/Omega 3 ratio. Group 2 allows affecting a value of the digestive inflammation.

In one embodiment, the value of Group 3 is determined using scores relating to a health questionnaire, a value relating to the measurement of the perception time, an homocysteine value, a value of the Omega 3 index, a score relating to cardiac variability parameters at night in particular, a value of the root mean square of the successive differences of the heart rate, a value of the LF/HF (low frequency/high frequency) ratio reflecting the sympathico-vagal overall balance, a value of the recovery index during sleep, and a value relating to measurements performed on the total activity of the autonomic nervous system. Group 3 allows affecting a value to the operation of the brain. In particular, measurement of the activity of the reptilian brain is performed through cardiac variability during effort and for 24 h which allows measuring sleep quality and an individual's capacity to recover. This measurement is essential in patients follow-up. The more one progresses in the program, the more the latter should improve.

In one embodiment, the value of Group 4 is determined in particular using scores relating to a health questionnaire, to a cardiac stress test in terms of speed or power, to the measurement of the heartbeat amplitude, to a score relating to a speed test at a value of the root mean square of the successive differences of the heart rate equal or lower than 10, to a somesthetic test, a visual test, a vestibular test, a ferritin value, a HOMA index, a Zinc value, a selenium value, a CRP value and a value of an immunity questionnaire. Group 4 allows affecting a value to the general health. Indeed, a complete assessment, focused on an in-depth functional neurology examination allows determining the operation level of the reptilian brain as well as the control capacity of the higher cortical areas. Moreover, individual operation as well as the interaction of the three main sensory systems, namely the peripheral, vestibular and ocular systems, are tested. The adaptation of the motor system, the visual capabilities when the head is moving as well as the functional capabilities of the joints of the lower limbs are also assessed. Preferably, each indicator has values comprised between 0 and 10.

For example the value of Group 1 may be determined according to the following formula:

Value Group 1=Σ(((3* Value digestive analysis)+(3* Value oxidized LDL)+2* (Value Zinc+Value Selenium)+Value HOMA index+Value Ferritin)*10)/12

The value of Group 2 may be determined according to the following formula:

Value Group 2=Σ(((5* Value CRP)+(3* Value digestive analysis)+2* (Value Omega6/Omega3)+Value HOMA index) *10)/11

The value of Group 3 may be determined according to the following formula:

Value Group 3=Σ(((3* Value health questionnaire)+(4* Value measurement of the perception time)+(5* Value measurements autonomic nervous system)+(3* Value cardiac variability parameters at night HF)+(3* Value cardiac variability parameters at night LF)+(3* Value root mean square of the successive differences of heart rate)+(2* Value homocysteine)+(Value LF/HF ratio)+Value Omega3 index+Value recovery index at sleep)*10)/26

Finally, the value of Group 4 may be determined according to the following formula:

Value Group 4=Σ(((3* Value health questionnaire)+(5* Value cardiac stress test speed)+(5* Value cardiac stress test power)+(4* Value heartbeat amplitude)+(4* Value speed test and value of the root mean square of the successive differences of the heart rate)+(2* Value somesthetic test)+(2* Value visual test)+(3* Value vestibular test)+(2* Value ferritin)+Value HOMA index+Value zinc+Value selenium+(4* Value CRP)+(2* Value immunity test))*10)/34

Optionally, besides tests composing the groups 1 to 4, calculation of the health score S takes into account two additional tests in the form of an urinalysis and of a genetic test allowing drawing up an assessment covering toxic metals, such as mercury, arsenic or lead so as to improve the health score of the patient. These additional tests do not aim to treat severe poisoning cases but to eliminate heavy metals from all those who suffer from chronic health problems and even those who think they are healthy and who have heavy metals in their body without knowing it.

In order to improve the state of health according to an obtained score, products and a program to follow are provided. In particular, these products and programs allow ensuring the proper operation of our two brains. Indeed, the work load of the digestive brain must be minimized as much as possible, also our flora and our intestinal mucosa must be preciously maintained. It is also essential to follow the rhythm of our reptilian brain, in particular by favoring the recovery period which takes place at night.

In a preferred embodiment, a product for its therapeutic use, includes between 1L and 3L of water, between 100 mL and 300 mL of lemon juice, between 100 mL and 300 mL of maple syrup, between 3 g and 200 g of ginger, between 1 g and 50 g of paprika, and between 1 g and 30 g of butyric acid. This product may be administered on an empty stomach for 2 to 6 days.

In another embodiment, a product for its therapeutic use, acting in particular on the elimination of detected heavy metals, includes between 5 g and 50 g of Chlorella, between 1 g and 20 g of Coriander and between 1 g and 20 g of Allium ursinum (bear's garlic). This product may be administered on an empty stomach for 2 to 6 days.

In another embodiment, a product for its therapeutic use by improving the state of health according to an obtained score includes between 300 mg and 800 mg of N-acetylcysteine, between 4 mg and 30 mg of Zincii (citras), between 10 mcg and 120 mcg of Selenium and between 100UI and 500UI of Chlocalciferolum D3.

In one embodiment, a product for its therapeutic use by improving the state of health according to an obtained score includes between 50 mcg and 400 mcg of Acidum folicum, between 0.1 mg and 5 mg of Methylcobalaminum, between 50 mg and 300 mg of S-adenosyl-L-methionine, and between 50 mg and 600 mg of Magnesii citras.

In one embodiment, a product for its therapeutic use by improving the state of health according to an obtained score includes between 10 mg and 400 mg of Resveratrolum, between 10 mg and 400 mg of Trigonella foenum graecum extractum siccum, between 10 mg and 400 mg of Acidum alginicum and between 10 mg and 400 mg of Lycopenum.

In one embodiment, a product in the form of biscuit for its therapeutic use by improving the state of health according to an obtained score includes between 1 g and 30 g of blond Psyllium and between 1 g and 30 g of oat bran.

In one embodiment, a product in the form of biscuit for its therapeutic use by improving the state of health according to an obtained score includes and by acting in particular on detected heavy metals, includes between 40% and 55% of oat bran, between 10% and 20% of apple Pectin, between 10% and 20% of Chlorella and between 10% and 20% of blond Psyllium.

The features of the invention will appear more clearly upon reading several embodiments given only as non-limiting examples, with reference to the schematic drawing, wherein:

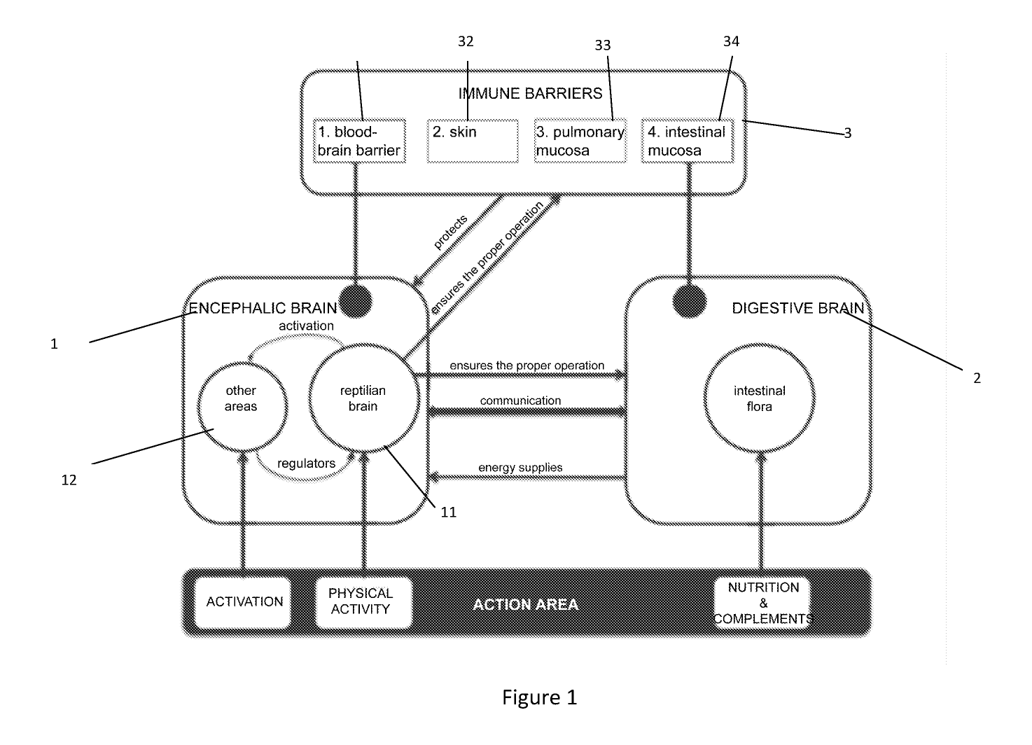

FIG. 1 is a diagram of the interaction between the encephalic brain and the digestive brain.

FIG. 1 illustrates the interaction between the encephalic brain 1 and the digestive brain 2. The encephalic brain 1 is composed of a brain called reptilian brain 11, which is the most important area of the encephalic brain 1 as regards health, and other areas 12 which have regulatory actions on the reptilian brain 11.

The digestive brain 2 must ensure energy supplies, essential nutrients, vitamins and trace elements. It must also act as a protective barrier in order to prevent the inlet of undesired intruders. The digestive brain 2 permanently communicates with the encephalic brain 1 via the enteric nervous system and by numerous hormones, lipoproteins, interleukins and other substances.

In order to improve our state of health, one must ensure a proper operation of the four immune barriers 3, namely the intestinal mucosa 34, the skin 32, the pulmonary mucosa 33 and the blood—brain barrier 31.

The four immune barriers 3 protect the encephalic brain 1. Moreover, one must also ensure the quality and the quantity of the nutritional supplies essential to the organism, the optimal activation of the encephalic 1 and reptilian 11 brains and finally an optimal operation of the higher centers of the brain for the regularization of the reptilian brain 11.

The encephalic brain 1 is the motor, regulator and coordinator of all the essential functions. For the encephalic brain 1 to operate well, it must on the one hand be protected by the digestive brain 2, and on the other hand it must be permanently activated. Physical activity, movement through the stimulation of the neurons of the cerebellum, is the most important activation. The other visual, auditory, gustatory and tactile stimulations also play this role.

For each patient, a complete functional assessment of both brains 1, 2 and of oxidative stress factors is performed in order to accurately determine a score of the state of health and of the individual objectives.

In order to determine the health score of a patient, the patient must undergo a series of tests, questionnaires which will allow determining a value to each indicator.

Thus, firstly, an establishment of the history of symptoms specific to the two brains 1, 2 allows quantifying and classifying dysfunctions according to their significance. A complementary questionnaire allows getting to know the dietary habits.

Afterwards, a clinical examination focused on an in-depth functional neurology examination allows determining the operation level of the reptilian brain 11 as well as the control capacity of the higher cortical areas.

In particular, the individual operation and the interaction of the three main sensory systems, namely the peripheral, vestibular and ocular systems, are assessed. The adaptation of the motor system, the visual capabilities when the head is moving and the functional capabilities of the joints of the lower limbs are also assessed.

Next, an examination of the locomotion functions and physical abilities allows determining the intensity of the physical activity which will be the most beneficial and the progress margin.

At a next step, a measurement of the bioimpedance determines the electrical resistance and the reactance of our body. These two values are important to monitor the evolution of the health of the patient. The estimates of the total body water, of the intra- and extra-cellular water are also important in the context of an individualized follow-up.

At one step, a micro-nutritional assessment comprises a specific blood panel allowing assessing the current oxidative stress, the capacities to deal with it, as well as the antecedent lesions that it has caused. This micro-nutritional assessment also comprises an assessment of fatty acids, a measurement of the digestive inflammation, if necessary a measurement of the intestinal permeability and finally a measurement of the capacity to produce energy and of the permeability of the blood-brain barrier.

Afterwards, the cardiac variability during effort and for 24h must be determined by a measurement of the activity of the reptilian brain through the cardiac variability during effort and for 24h. This test also allows to measure the sleep quality and the individual capacity to recover.

Finally, a step of measuring the force of the inspiratory muscles allows to assess the operation of the thoracic cage as well as the presence of metaboreflex, which is an important sign of hyperactivity of the sympathetic system.

Once all of these steps are carried out, the measured values of the indicators allow establishment for each of said indicators, a score depending on the measured value with respect to reference values.

A value is assigned to each of the four groups constituted by said indicators, each of said groups representing information corresponding respectively to the oxidative stress, hereinafter Group 1; to the functions of the digestive brain, hereinafter Group 2; to the functions of the reptilian brain, hereinafter Group 3; and to the physical abilities of the patient coupled to his information on his general state of health, hereinafter Group 4.

Finally, a health score S specific to the patient is calculated, on a scale ranging from 0 to 100, using the formula:

S=(Value Group 1+(2* Value Group 2)+(2* Value Group 3)+(3* Value Group 4))/(2* Number of groups)

For example, a patient whose value of group 1 is 49.17, value of group 2 is 47.27, value of group 3 is 86.92 and value of group 4 is 99.12 would have a health score S=76.86.

In this example, the closer the value of the health score is to the value 100, the more the patient is in good health.

In order to improve this score, the patient can take a drink including for example 2L of water, 150 mL of lemon juice, 150 mL of maple syrup, 2 g of ginger, 1 g of paprika and 10 g of butyric acid.

This drink can be administered exclusively (on an empty stomach) for a period of 4 days for example.

If the patient has performed the two additional tests in the form of an urinalysis and of a genetic test allowing drawing up an assessment covering toxic metals, such as mercury, arsenic or lead so as to improve the health score of the patient, depending on the measured results, he can ingest, on a daily basis, a product to improve the result, the product including for example 25 g of chlorella, 5 g of coriander, and 5 g of allium ursinum (bear's garlic). This product may also be ingested for about 4 days.

Still to improve the measured health score, the patient can ingest, in the form of pills, a product including 600 mg of N-acetylcysteine, 12 mg of Zincii (citras), 50 mcg of Selenium, 300UI of Chlocalciferolum D3.

In the same manner, to improve the measured health score, the patient can ingest, in the form of pills, a product including 200 mcg of acidum folicum, 1 mg of methylcobalaminum, 100 mg of S-adenosyl-L-methionine and 250 mg of magnesii citras.

In order to improve the measured health score, the patient can ingest, in the form of pills, another product including 100 mg of Resveratrolum, 250 mg of Trigonella foenum graecum extractum siccum, 250 mg of Acidum alginicum, and 250 mg of Lycopenum.

In the long run and in order to stabilize achievements having allowed reaching a better result, a product in the form of biscuit including 5 g of blond psyllium and 5 g of oat bran may be ingested every day.

In order to tackle heavy metals over time, another product in the form of a biscuit may be ingested by the patient, the biscuit including 50% of oat bran, 16.6% of apple pectin, 16.6% of Chlorella and 16.6% of blond Psyllium.

For example, a four-month period allows establishing significant changes having a real impact on health. It is only at the end of that period that the improvement of the blood panel and of the sleep quality will be visible, as best indicator of the function of the reptilian brain of the patient. The comparative analysis of the assessments before and after the program will demonstrate the improvement on the health score of the patient.

1. A method for determining a numerical score representative of a patient's health, characterized by the following steps:
at an initial calibration step, a database is established from a series of indicators relating to the state of health of the patient, each indicator being assigned a numerical value, comprised between 0 and 10, and afterwards
a statistical analysis of this database is performed so as to:
establish for each of said indicators a score, comprised for example between 1 and 10, depending on a measured value of the indicators of the state of health with respect to reference values,
establish at least four groups constituted by said indicators, each of said groups representing information corresponding respectively: to oxidative stress, hereinafter Group 1; to functions of the digestive brain, hereinafter Group 2; to functions of the reptilian brain, hereinafter Group 3; and to physical abilities of the patient coupled to his information on his general state of health, hereinafter Group 4, and
a value is assigned to each group, comprised between 0 and 100, the value of each group being derived from the results of each indicator, then a health score S specific to the patient is calculated using the formula:
S=Σ(Value Group 1+(2* Value Group 2)+(2* Value Group 3)+(3* Value Group 4))/(2* Number of groups)

2. The method according to claim 1, characterized in that the numerical score is determined on a scale of 0 to 100.

3. The method according to claim 1, characterized in that the value of Group 1 is determined using scores relating to a digestive analysis, an oxidized LDL (Low Density Lipoprotein) value, a HOMA (homoestasis model assessment) index, a Selenium value, a Ferritin value, and a Zinc value.

4. The method according to claim 1, characterized in that the value of Group 2 is determined using scores relating to a digestive analysis, a CRP (C-reactive protein) value, a HOMA index and a value of the ratio Omega 6/Omega 3.

5. The method according to claim 1, characterized in that the value of Group 3 is determined using scores relating to a health questionnaire, a value relating to the measurement of the perception time, an homocysteine value, a value of the Omega 3 index, a score relating to cardiac variability parameters at night in particular, a value of the root mean square of the successive differences of the heart rate, a value of the LF/HF (low frequency/high frequency) ratio reflecting the sympathico-vagal overall balance, a value of the recovery index during sleep, and a value relating to measurements performed on the total activity of the autonomous nervous system.

6. The method according to claim 1, characterized in that the value of Group 4 is determined in particular using scores relating to a health questionnaire, to a cardiac stress test in terms of speed or power, to the measurement of the heartbeat amplitude, to a score relating to a speed test at a value of the root mean square of successive differences of the heart rate equal or lower than 10, to a somesthetic test, a visual test, a vestibular test, a ferritin value, a HOMA index, a Zinc value, a selenium value, a CRP value and a value of an immunity questionnaire.

7. The method according to claim 1, characterized in that values comprised between 0 and 10 are assigned to each indicator.

8. The method according to claim 1, characterized in that besides the tests composing groups 1 to 4, calculation of the health score S takes into account two additional tests in the form of an urinalysis and a genetic test allowing drawing up an assessment covering toxic metals, such as mercury, arsenic or lead.

9. A product for its therapeutic use by improving the state of health according to a score obtained by the method according to claim 1, the product including between 1L and 3L of water, between 100 mL and 300 mL of lemon juice, between 100 mL and 300 mL of maple syrup, between 3 g and 200 g of ginger, between 1 g and 50 g of paprika and between 1 g and 30 g of butyric acid.

10. A product for its therapeutic use by improving state of health according to a score obtained by the method of claim 1, by acting in particular on the detected heavy metals, the product including between 5 g and 50 g of Chlorella, between 1 g and 20 g of Coriander and between 1 g and 20 g of Allium ursinum (bear's garlic).

11. The product according to claim 9, intended to be administered on an empty stomach for 2 to 6 days.

12. A product for its therapeutic use by improving the state of health according to a score obtained by the method of claim 1, the product including either:
a. Between 300 mg and 800 mg of N-acetyl-cysteine, between 4 mg and 30 mg of Zincii (citras), between 10 mcg and 120 mcg of Selenium and between 10001 and 50001 of Chlocalciferolum D3; or
b. Between 50 mcg and 400 mcg of Acidum folicum, between 0.1 mg and 5 mg of Methylcobalaminum, between 50 mg and 300 mg of S-adenosyl-L-methionine, and between 50 mg and 600 mg of Magnesii citras; or
c. Between 10 mg and 400 mg of Resveratrolum, between 10 mg and 400 mg of Trigonella foenum graecum extractum siccum, between 10 mg and 400 mg of Acidum alginicum and between 10 mg and 400 mg of Lycopenum.

13. A product for its therapeutic use by improving the state of health according to a score obtained by the method of claim 1, the product in the form of biscuit including between 1 g and 30 g of blond Psyllium and between 1 g and 30 g of oat bran.

14. A product for its therapeutic use by improving the state of health according to a score obtained by the method of claim 1, by acting in particular on detected heavy metals, the product in the form of biscuit including between 40% and 55% of oat bran, between 10% and 20% of apple Pectin, between 10% and 20% of Chlorella and between 10% and 20% of blond Psyllium.
